(19) United States
(12) Patent Application Publication
(10) Pub. No.: US 2026/0083108 A1
(43) Pub. Date: Mar. 26, 2026

(54) AN INSECT REARING/BREEDING HOUSING

(71) Applicant: SKOV A/S, Roslev (DK)

(72) Inventors: Lars-Henrik Lau HECKMANN, Roslev (DK); Toke Munk SCHOU, Flemming (DK); Michael Hyldig CHRISTENSEN, Roslev (DK); Arne Blok LAURIDSEN, Flemming (DK); Henrik VOIGT, Roslev (DK); Carsten Lind PEDERSEN, Flemming (DK)

(73) Assignee: SKOV A/S, Roslev (DK)

(21) Appl. No.: 19/109,244

(22) PCT Filed: Aug. 28, 2023
(86) PCT No.: PCT/EP2023/073513

§ 371 Date: Mar. 6, 2025

(30) Foreign Application Priority Data
Sep. 14, 2022 (DK) PA202270443

(51) Int. Cl. A01K 67/362; A01K 67/364

(52) U.S. Cl. CPC A01K 67/362; A01K 67/364

(57) ABSTRACT

An insect rearing/breeding housing, wherein a plurality of crates arranged in a row along the length of the housing define at least a portion of the housing bottom. Holes formed in the bottom of the crates allow for air to be sucked into the housing and to be discharged through at least one air ventilating opening of the housing. The housing having a crate insertion opening in at least one end and a crate discharge opening at the housing bottom. The crates being supported so as to be movable from said insertion opening to the discharge opening.

### TECHNICAL FIELD

The present disclosure relates to an insect rearing/breeding housing. More specifically, the disclosure relates to an insect rearing/breeding housing as defined in the introductory parts of the independent claims.

### BACKGROUND ART

In known insect rearing/breeding housings for example as mentioned in WO2020245158 A1 a number of crates are placed in a rearing/breeding housing in stacks. A problem with the solutions of the prior art is that providing proper and uniform ventilation or conditioned air to each crate in a housing for rearing insect larvae is a challenge and needs dedicated nozzles for the individual crates placed in the housing. There is thus a need for improved equipment for rearing/breeding insects.

### SUMMARY

It is an object of the present disclosure to mitigate, alleviate or eliminate one or more of the above-identified deficiencies and disadvantages in the prior art and solve at least the above-mentioned problem. According to a first aspect, there is provided an insect rearing/breeding housing, wherein a plurality of crates arranged in a row along the length of the housing define at least a portion of the housing bottom, and wherein holes formed in the bottom of the crates allow for air to be sucked into the housing and to be discharged through at least one air ventilating opening of the housing, the housing having a crate insertion opening in at least one end and a crate discharge opening at the housing bottom, the crates being supported so as to be movable from the insertion opening to the discharge opening.

Hereby it is possible to provide an even ventilation or air stream through the crates and the housing.

According to some embodiments, the crate discharge opening is provided at an opposite end in relation to a crate insertion opening at one end and at the housing bottom,

Hereby is achieved that crates can be inserted into the rearing/breeding housing in one end of the housing and can be removed from the opposite end of the housing.

According to some embodiments, the housing is provided with a crate insertion opening at both ends of said housing and a discharge opening is provided at a middle portion of the housing at the housing bottom.

Hereby is achieved that crates can be inserted in both ends of the housing and that crates can be removed from a middle part of the housing.

According to some embodiments, longitudinal support flanges for supporting the crates is provided at each side of the housing extending from the crate insertion opening to the crate discharge opening.

Hereby is achieved a connection between the crates and the housing where the crates can be moved through the housing from the crate insertion opening in at least one end and towards the crate discharge opening and at the same time provide a connection preventing the flying insects from escaping the housing since the flanges and the crates forms a connection where an edge of the crate slide on an upper side of the flanges at each side of the housing.

According to some embodiments, a longitudinal flange is provided at each side of the housing, parallel to the support flanges and having a distance from the support flanges corresponding to a height of an edge of the crates in such a way that the edge of the crates can be moved along the support flanges.

According to some embodiments, the longitudinal support flanges for supporting the crates provided at each side of the housing, extends from the crate insertion openings at each end of the housing to the crate discharge opening provided at a middle portion of the housing at the housing bottom.

Hereby is achieved that the edges of crates abutting each other when moved from the crate insertion opening to the crate discharge opening, are aligned and do not tend to be displaced in height in such a way that the edges will form an air gap in one end relative to the flanges.

The further longitudinal flanges are preferably placed above the support flanges.

According to some embodiments, the longitudinal support flanges leaves a gap at each side of the housing, for removal of the crates.

Hereby it is possible to remove crates by lowering the crates and thereby remove crates from the housing bottom.

According to some embodiments, a cover plate is provided above and between the gaps, which cover plate slopes downwards to at least one side along a moving direction of the crates.

Hereby is achieved that dead flies or the like will fall into the crates and thereby removed by the discharged crates to avoid dead flies or the like generating smells attracting flies to lay eggs different places than in the egg module.

According to some embodiments, a separating wall or plate extends from a top part of the cover plate to the top of the housing, separating the housing in two.

This makes it possible for rearing/breeding in two different populations in the same housing, still keeping the populations separated from each other.

According to some embodiments, the cover plate is provided with a sealing for sealing towards an upper edge of the crates.

Hereby is achieved that insects are prevented from escaping the housing when discharging crates from the discharge opening.

According to some embodiments, ventilating air is supplied through the bottom of the housing from below the crates.

This is an advantage since it is possible to have a housing with an open bottom structure, where the bottom opening is closed by crates with a plurality of perforations in the bottom of the crates, which crates are used for housing the pupae. Hereby it is also possible to remove smells from i.e. pupae remains or the like, from the housing.

According to some embodiments, ventilating air is sucked out at top of the housing by a fan or blower.

Hereby it is possible to suck out air from the housing or to assist removing air by blowers, fans or the like, which air may be forced into the housing by blowers, fans or the like or just sucked into the housing through the bottom and out through top openings.

According to some embodiments, ventilating air is supplied through one or more openings at a top part of the housing.

Hereby is disclosed an alternative way of supplying ventilating air to the housing.

To further enhance ventilating flow through the crates, a kind of curtains are provided extending along sides of the crates from the longitudinal support flanges and to at least an underside of the crates. This ensures a directional flow of air, which is thereby forced or guided to primarily go up through the bottom of the crates and not partially through the holes in the sides of the crates.

The curtains can be made of metal, plastic or another suitable substantially rigid material that will not flap under the influence of the ventilation air.

In a simple embodiment, the curtains are formed by the sides of the housing, which sides continues from the top of the housing to below or to a point level with the underside of the crates when placed in the housing.

According to some embodiments, ventilating air is alternating between being supplied through one or more openings at a top part of the housing and through the bottom from below the crates.

Hereby is disclosed yet an alternative way of supplying ventilating air to the housing. Alternating the air stream may clear openings or perforations in the bottom of the crates when covered by remains from the pupae.

According to some embodiments, one or more ventilating openings at top of the housing are provided with a net or grate, preventing flies from entering the one or more ventilating openings.

Hereby is prevented that flying insects, for example flies can escape the housing by entering the one or more ventilating openings.

According to some embodiments, an inspection window is provided in at least one end of the housing.

Hereby it is possible to follow the course of the rearing/breeding process to be able to intervene if necessary.

According to some embodiments, crate insertion openings and crate discharge openings are provided with a sealing for sealing towards an upper edge of the crates.

Hereby is also achieved that insects are prevented from escaping the housing when discharging crates from the discharge opening.

According to some embodiments, a conveyor is provided at the discharge opening for conveying discharged crates in a crosswise direction in relation to movement of the crates in the housing.

According to some embodiments, the conveyor is placed below the crate discharge opening at a middle portion of the housing at the housing bottom.

Providing a conveyor to handle discharged crates from the housing gives possibilities for building of a more flexible rearing/breeding system comprising a plurality of rearing/breeding housings placed next to each other.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows a rearing/breeding housing where a row of crates is inserted in the bottom according to an embodiment of the present disclosure;

FIG. 2 shows an empty rearing/breeding housing. according to an embodiment of the present disclosure;

FIG. 3 shows a front view of a rearing/breeding housing where a row of crates is inserted in the bottom according to an embodiment of the present disclosure;

FIG. 4 shows an empty rearing/breeding housing seen from the insertion end from where crates can be inserted according to an embodiment of the present disclosure;

FIG. 5 shows schematically a possible ventilating flow in a rearing/breeding housing according to the embodiment in FIG. 1;

FIG. 6 shows a rearing/breeding housing where a row of crates is inserted in the bottom according to an alternative embodiment of the present disclosure;

FIG. 7 shows the embodiment of FIG. 6 where a side is removed;

FIG. 8 shows an embodiment where the housing is separated in two rearing/breeding spaces;

FIG. 9 shows schematically an alternative embodiment provided with a conveyor for transport of discharged crates; and

FIG. 10 shows schematically a facility comprising a plurality of rearing/breeding housings.

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

The insect rearing/breeding housing 1 can be used for rearing/breeding of various kinds of flying insects.

In the following an insect rearing/breeding housing 1 for Black Soldier Fly hereafter called BSF, but not limited hereto, will be described.

When BSF larvae mature, they grow into approx. 12 mm long grubs, a point (after approximately 2-3 weeks) at which they become pre-pupa and finally turn into pupae at which condition they may be up to 25-30 mm long. The BSF larvae can be fed to for example fish, poultry and pigs and are a good source of protein. It is also possible to dry the larvae to be processed into feed for use at a later time.

Following minimum one week of maturation, the pupae emerge as flies that breed, propagating the population.

In this way it is possible to produce enough flies for maintaining the population as well as growing larvae to provide feed to i.e. chickens or other live animals.

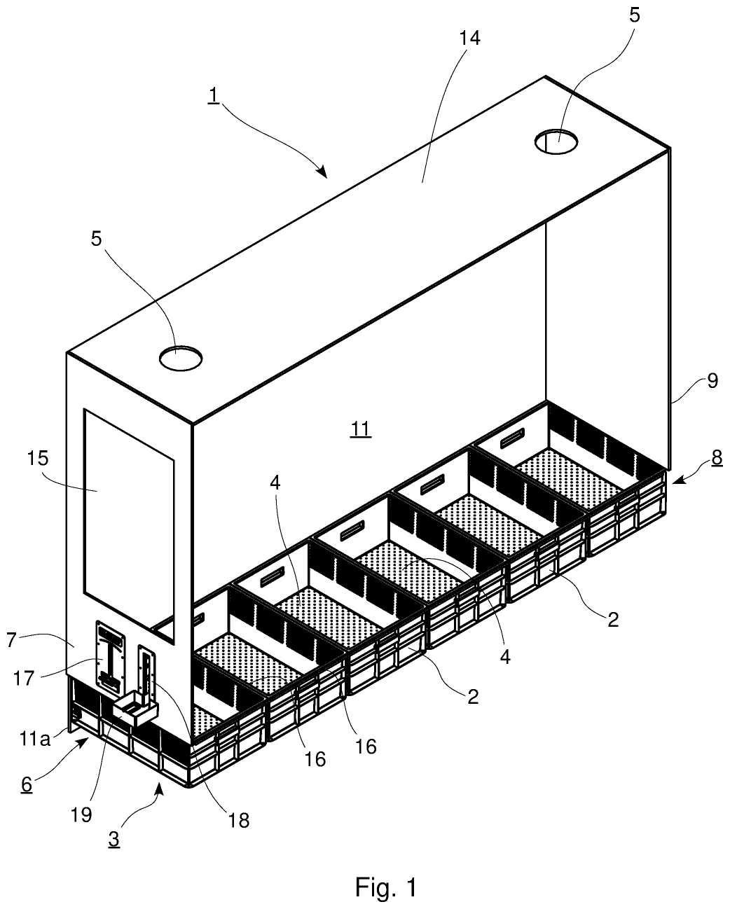

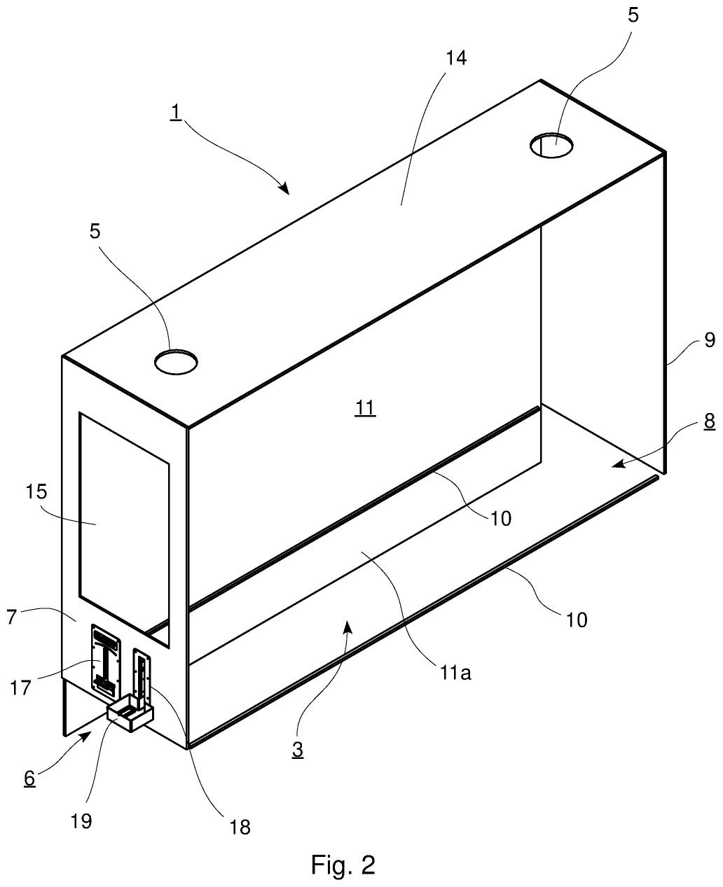

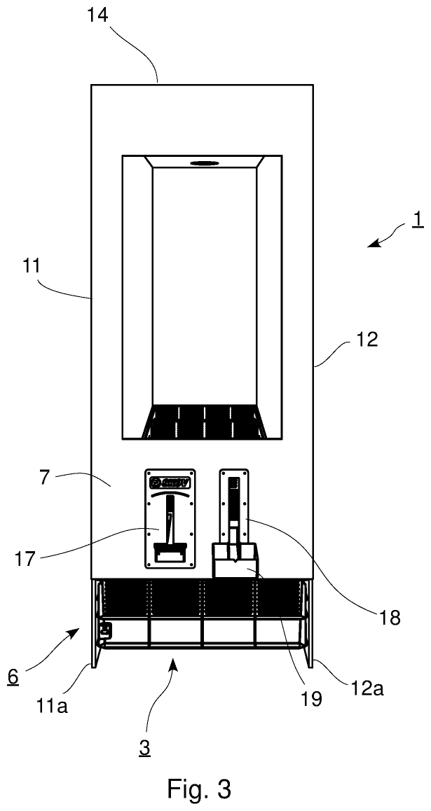

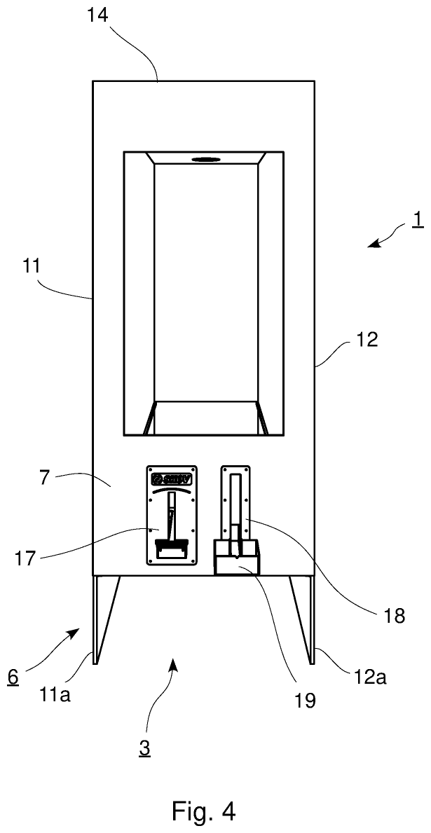

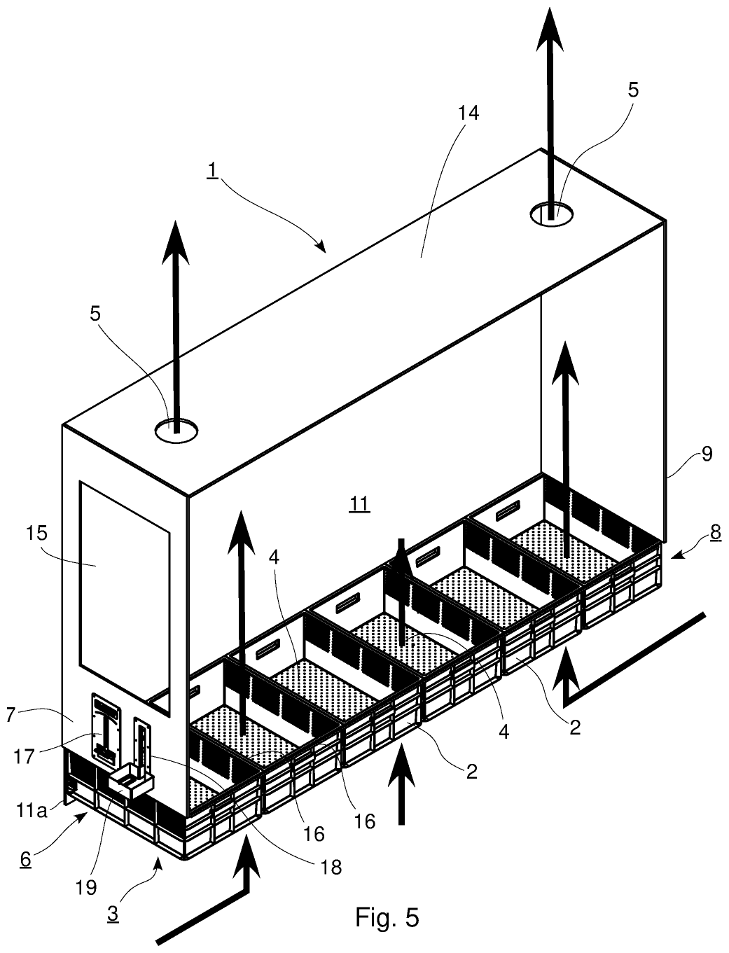

### FIGS. 1 to 5 shows a rearing/breeding housing 1 according to a first aspect of the disclosure.

The first aspect of this disclosure shows an insect rearing/breeding housing 1, wherein a plurality of crates 2 arranged in a row along the length of the housing 1 define at least a portion of the housing bottom 3, and wherein holes 4 formed in the bottom of the crates 2 allow for air to be sucked into the housing 1 and to be discharged through at least one air ventilating opening 5 of the housing 1, the housing 1 having a crate insertion opening 6 at one end 7 and a crate discharge opening 8 at an opposite end 9, the crates 2 being supported so as to be movable from the insertion opening 6 to the discharge opening 8.

In an embodiment of the crates 2, holes are provided in sides and/or ends of the crates 2, at least in an upper part of the sides and/or ends of the crates 2. This allows for ventilation both through bottom holes 4 and through sides and/or ends of the crates 2. Hereby is achieved an enhanced ventilation flow through the crates when placed in the housing bottom 3 of the insect rearing/breeding housing 1, and even when the crates are stacked, waiting to be inserted in the insect rearing/breeding housing 1.

In FIGS. 1, 3 and 5 the rearing/breeding housing 1 shows a row of crates 2 inserted through a crate insertion opening 6 in one end 7 of the housing 1 whereby the crates 2 forms or defines at least a portion of the housing bottom 3.

When starting the rearing or breeding process, the process can advantageously be started by pushing a number of empty crates 2 as well as a number of crates containing pupae into the bottom 3 of the housing 1.

For example, in a housing 1 where the bottom 3 is formed by six crates 2 the first four crates 2 can be empty and the last two crates 2 contains pupae for breeding.

Over the course of three to six days, the pupae develop legs and wings, ultimately emerging as full-grown flies. Within two to three days, female flies are capable of reproduction and to lie eggs, which eggs hatch into larvae and the larvae pupate.

To fit into the breeding process of the insects, the crates are moved in the housing adapting to the process. After a number of days, a further number of crates 2 are inserted in the bottom 3 of the housing 1 whereby an identical number of empty crates are pushed out at the opposite end 9 of the housing 1. After yet a number of days same procedure is followed where after the bottom 3 of the housing 1 is filled with pupae containing crates 2.

Then this sequence continues rearing/breeding the insects and hereby crates 2 where pupae has turned into flies are removed from discharge opening 8 at the backend 9 of the housing 1 in pace with new crates 2 being supplied through the insertion opening 6 at the front end 7 of the housing.

In the bottom 3 of the housing, longitudinal support flanges 10 for supporting the crates 2 is provided at each side 11, 12 of the housing 1 extending from the crate insertion opening 6 to the crate discharge opening 8. The crates 2 can by the edges of the underside of the crates 2 simply support on the flanges 10, or the crates can be formed with an upper edge 16 extending perpendicular from the sides of the crates in a substantially horizontal direction. The edge does not need to be at the upper end of the sides of the crates 2, what is important is that the edge 16 extends from the side of the crate in such a way that the edge 16 can bear on the longitudinal support flanges 10.

In an aspect, a further longitudinal flange is provided at each side 11, 12 of the housing 1, parallel to the support flanges 10 and having a distance from the support flanges 10 corresponding to a height of an edge 16 of the crates 2.

Hereby the crates are prevented from being displaced in a vertical direction when pushed from the inlet opening 6 to the outlet opening 8 and ensures that insects are prevented from escaping the housing 1 through gaps between the edges 16 of the crates 2 and the support flanges 10.

The further longitudinal flanges are preferably placed above the support flanges 10.

In an aspect, ventilating air is supplied through the bottom 3 of the housing 1 below the crates 2. This is possible due to a number of perforations or holes 4 formed in the bottom of the crates 2.

In an aspect, ventilating air is sucked out at top 14 of the housing 1 by a fan or blower (not shown).

In an aspect, ventilating air is supplied through one or more openings 5 at a top part 14 of the housing 1.

In an aspect, ventilating air is alternating between being supplied through one or more openings 5 at a top part 14 of the housing 1 and through the bottom 3 below the crates 2.

To further enhance ventilating flow through the crates 2, a kind of curtains 11a, 12a are provided extending along sides of the crates 2 from the longitudinal support flanges 10 and to at least an underside of the crates 2. This ensures a directional flow of air, which is thereby forced or guided to primarily go up through the holes 4 in bottom of the crates 2 and not partially through the holes in the sides of the crates 2.

The curtains 11a, 12a can be made of metal, plastic or another suitable substantially rigid material that will not flap under the influence of the ventilation air.

In a simple embodiment, the curtains 11a, 12a are formed by the sides 11, 12 of the housing 1, which sides 11, 12 continues from the top 14 of the housing 1 to below or to a point level with the underside of the crates 2 when placed in the housing 1.

In an aspect, one or more ventilating openings 5 at top 14 of the housing 1 are provided with a net or grate (not shown), preventing flies from entering the one or more ventilating openings 5.

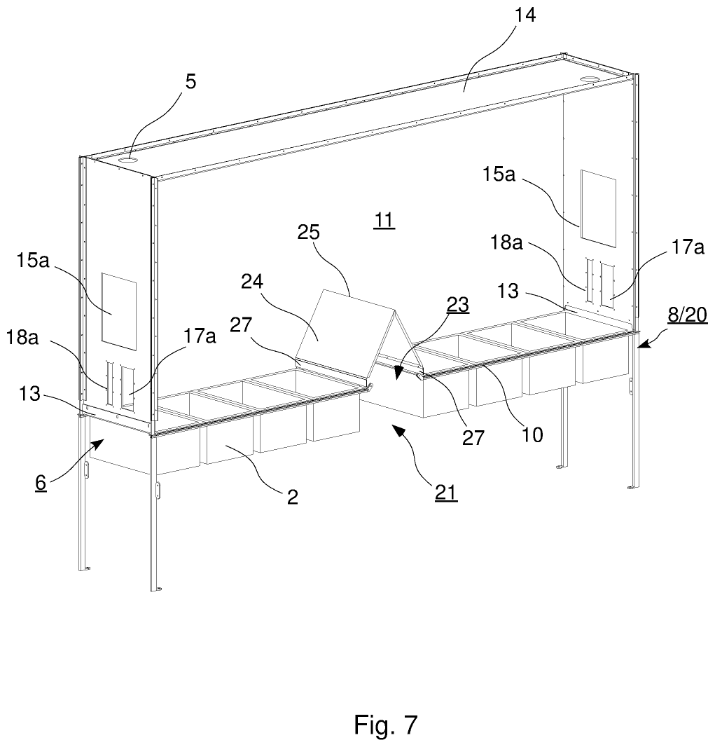

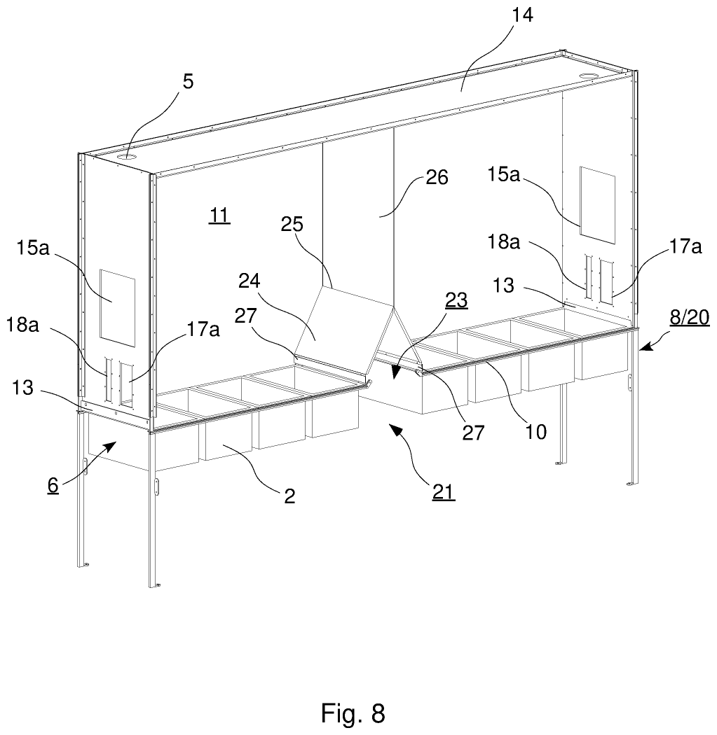

In an aspect, an inspection window 15 is provided at an end 7, 9 of the housing 1. For convenient reasons the inspection window 15 is placed at the end of the housing from where it is operated, normally at the insertion end 7 of the housing 1, but in some embodiments, a further inspection window can be placed at the outlet end 9 or even in both ends 7, 9 as indicated by openings 15a in FIGS. 7 and 8.

### Hereby it is possible to follow if the rearing/breeding process proceeds as planned.

In an aspect, the crate insertion opening 6 at one end 7 and the crate discharge opening 8 at the opposite end 9 of the housing 1 are provided with a sealing 13 for sealing towards an upper edge 16 of the crates 2. This sealing prevents insects from escaping the housing when inserting and removing crates.

The crates 2 are also formed in such a way that the edges on sides of the crates 2 abutting each other, leaving no gaps for insects to escape between the crates 2 forming the bottom 3 of the housing 1.

The egg module 17 is placed on an end 7 of the rearing/breeding housing 1 comprising the insertion opening 6 for the crates 2. The egg module 17 can be placed and replaced from outside the housing and comprises a cardboard hole or cell structure of a type similar to a honeycomb structure of a honey frame for harvesting honey from bees. The egg module 17 also comprises a bait box for emitting a homey smell or lure scent, to attract the flying insects, here female Black Soldier Flies, and encourage the flies to lay eggs in the cells in the egg module 17. When the cells are sufficiently filled with eggs, the eggs are harvested by removing the cardboard structure from the egg module 17 and a new cardboard structure is inserted for further harvesting of eggs. In an embodiment, the whole egg module, including the cardboard structure, can be replaced, when harvesting.

In an embodiment the egg module 17 can be made of other materials, which for example include materials based on plastic or recycled plastic, thereby achieving a greater degree of reusability of egg modules 17 in the housing 1.

Also close to the egg module 17 in the same end 7 of the rearing/breeding housing 1 a water module 18 is placed. The water module 18 provides drinking water to the flies and is configured with a water reservoir 19 at a bottom part of the module, which reservoir 19 extends from outside the housing 1 and into the housing 1. At the side of the water module 18 extending on the inner side of the front end 7 of the housing 1, a porous material due to capillary effect, distributes the water from the reservoir to the surface of the material so that the flies can settle on the material and drink.

The material can be a porous stone or another suitable porous material.

Placing the water module 18 close to the egg module 17 also attracts the flies to the area where you want them to lay their eggs.

The water module can in an embodiment be connected to an external water supply, such as an industrial water supply or a drinking water supply for automatically filling the reservoir, when emptied.

Ventilation of the rearing/breeding housing 1 is necessary due to the fact that insects and especially flies are attracted by strong smells of spoiled things, such as rotten meat or rotten fruits or the like. Hence, the remains from the pupae when turning into flies as well as fly droppings in huge numbers will attract the flies and eventually the smell will drown out the smell from the bait box. Therefore, it is important that the ventilation removes the smell from the crates 2 so that the flies are still attracted to the egg module 17 for laying eggs. Laying eggs in the crates 2 will cause that a huge number of eggs will be extremely difficult to harvest and thereby lost.

To encourage the flies to lay their eggs in the egg module 17, a sufficient level of ventilation ensures, in combination with removal of old crates 2, that a clean environment is maintained within the rearing/breeding housing 1. This ensures that the units can be operated continuously, at least in periods of 2 to 4 weeks, which is cost-efficient.

A sufficient air exchange can be at least 20 times the volume of the rearing/breeding housing 1 per hour. It is also necessary to ensure that the ventilation does not blow remains from pupated pupae around in the housing.

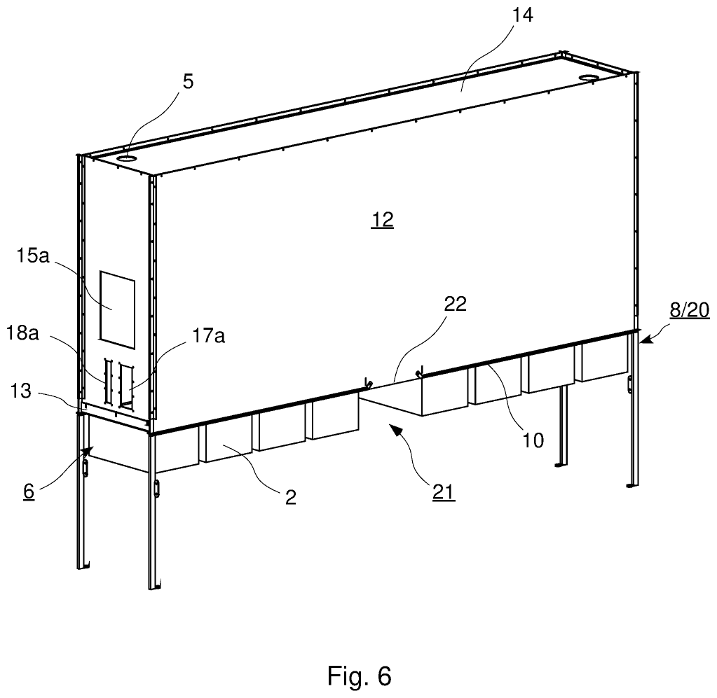

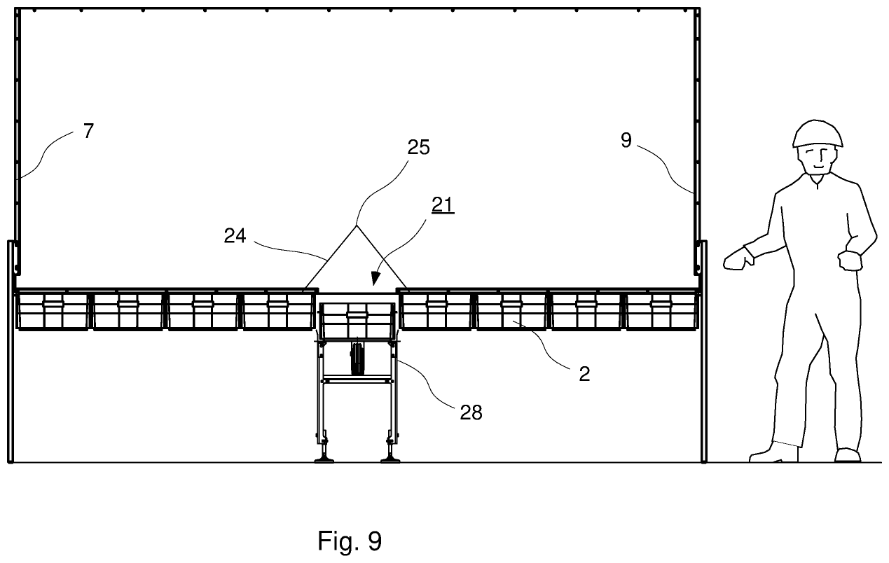

FIG. 6 to 10 shows a rearing/breeding housing 1 according to a second aspect of the disclosure.

In embodiments shown in FIGS. 6-10 an egg module 17 and a water module 18 can be provided at each end 7, 9 as indicated by openings 17a and 18a in FIGS. 7 and 8.

In an aspect, the housing 1 is provided with a crate insertion opening 6, 20 at both ends of said housing 1 and a discharge opening 21 is provided at a middle portion 22 of the housing 1 at the housing bottom 3.

In an aspect, the longitudinal support flanges 10 for supporting the crates 2 provided at each side 11, 12 of the housing 1, extends from the crate insertion openings 6, 20 at each end of the housing 1 to the crate discharge opening 21 provided at a middle portion 22 of the housing 1 at the housing bottom 3.

In an aspect, the longitudinal support flanges 10 leaves a gap 23 at each side 11, 12 of the housing 1, for removal of the crates 2.

In an aspect, a cover plate 24 is provided above and between the gaps 23, which cover plate 24 slopes downwards to at least one side along a moving direction of the crates 2. The sloping sides provides that dead flies or the like will fall into the crates 2 and are thereby removed by the discharged crates 2 to avoid dead flies or the like generating smells attracting flies to lay eggs different places than in the egg module 17.

In an aspect, a separating wall or plate 26 extends from a top part 25 of the cover plate 24 to the top 14 of the housing 1, separating the housing 1 in two.

Separating the housing 1 in two makes it possible for rearing/breeding in two different enclosures in the same housing 1, and thereby keeping the populations separated from each other.

In an aspect, the cover plate 24 is provided with a sealing 27 for sealing towards an upper edge 16 of the crates 2.

In an aspect, the cover plate 24 is provided with a sealing 27 at each side for sealing towards an upper edge 16 of the crates 2 to prevent insects from escaping the housing 1 when discharging crates 2 from the discharge opening 8, 21.

In an aspect, a conveyor 28 is provided at the discharge opening 8, 21 for conveying discharged crates 2 in a crosswise direction in relation to movement of the crates 2 in the housing 1.

In an aspect, the conveyor 28 is placed below the crate discharge opening 21 at a middle portion 22 of the housing 1 at the housing bottom 3.

Providing a conveyor 28 to handle discharged crates 2 from the housing 1 gives possibilities for building of a more flexible rearing/breeding system 29 comprising a plurality of rearing/breeding housings 1 placed next to each other.

The conveyor 28 can be placed on a supporting frame or another kind of known supports for conveyors 28.

In the aspects where the longitudinal support flanges 10 leaves a gap 23 at each side 11, 12 of the housing 1, for removal of the crates 2, a similar gap in curtains 11a, 12a, provided extending along sides of the crates 2 from the longitudinal support flanges 10 and to at least an underside of the crates 2 may be provided to let the crates 2 be transported on the conveyor 28.

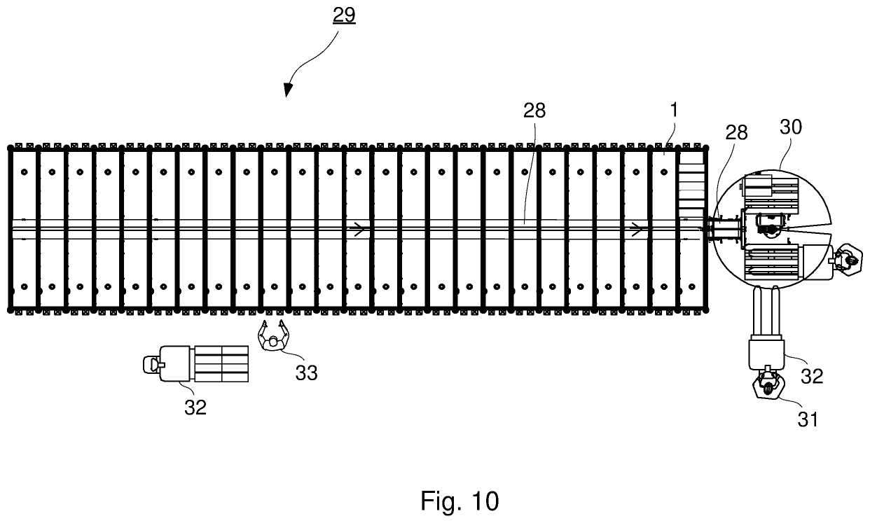

FIG. 10 shows a rearing/breeding system 29 comprising a plurality of rearing/breeding housings 1 placed next to each other and with a conveyor 28 placed in a crosswise direction in relation to a moving direction of the crates 2 in the housings 1.

At one end of the conveyor 28 from where crates 2 form the housings are discharged a handling area is provided where an operator 31 can remove discharges crates 2 by means of a pallet lifter, a forklift or similar transport equipment 32.

An operator 33 for harvesting eggs from the egg modules, operates along the ends 7, 9 of the plurality of housings 1 and can place the harvested eggs on pallets, crates, baskets or the like and transport them by means of a pallet lifter, a forklift or similar transport equipment 32.

In an aspect one or more of the operators 31, 33 can be assisted or replaced by a robot or other automated equipment for handling the egg modules 17 or crates 2.

In an aspect the housings 1 are placed on supporting legs or frames for supporting the housings 1 in a distance above a floor or above the ground in a building or similar.

In another aspect, the housings 1 are supported by support means (not shown), such as wires, stays, rods or the like, which hang down from a ceiling other support structure situated above the housings 1, such that the housings 1 hang freely over a floor or similar. This makes it easier to clean under the houses.

The person skilled in the art realizes that the present disclosure is not limited the preferred embodiments described above. The person skilled in the art further realizes that modifications and variations are possible within the scope the appended claims. For example, egg modules and water modules can be placed different places what is important is that it is possible for an operator to exchange the modules, refill water and harvest the eggs. Additionally, variations to the disclosed embodiments can be understood and effected by the skilled person in practicing the claimed disclosure, from a study of the drawings, the disclosure, and the appended claims.

1. An insect rearing/breeding housing comprising:
a plurality of crates arranged in a row along a length of the housing and defining at least a portion of a housing bottom, where the housing bottom is closed by the plurality of crates and wherein holes formed in a bottom of the plurality of crates allow for air to be sucked into the housing and to be discharged through at least one air ventilating opening of the housing;
a crate insertion opening in an end of the housing; and
a crate discharge opening at the housing bottom, the plurality of crates being supported so as to be movable from the insertion opening to the discharge opening.

2. The insect rearing/breeding housing according to claim 1, wherein the crate discharge opening is provided at an opposite end of the housing in relation to the crate insertion opening in one end and at the housing bottom.

3. The insect rearing/breeding housing according to claim 1, wherein the housing is provided with a second crate insertion opening at a second end of the housing and wherein the crate discharge opening is provided at a middle portion of the housing at the housing bottom.

4. The insect rearing/breeding housing according to claim 2, wherein a first longitudinal support flange and a second longitudinal support flange for supporting the plurality of crates are provided at a first side of the housing and a second side of the housing, respectively, extending from the crate insertion opening to crate discharge opening.

5. The insect rearing/breeding housing according to claim 4, wherein the first longitudinal support flange is provided at the first side of the housing and the second longitudinal support flange is provided at the second each side of the housing, wherein the first side of the housing is parallel to the first longitudinal support flange and having a distance from the first longitudinal support flange corresponding to a heigh of an edge of the plurality of crates and the second side of the housing is parallel to the second longitudinal support flange and having a distance from the second longitudinal support flange corresponding to the height of the edge of the plurality of crates.

6. The insect rearing/breeding housing according to claim 3, wherein a first longitudinal support flange and a second longitudinal flange for supporting the plurality of crates are provided at a first side of the housing and a second side of the housing, respectively, and extend from the crate insertion opening and the second crate insertion opening at the end of the housing and the second end of the housing to the crate discharge opening provided at the middle portion of the housing at the housing bottom.

7. The insect rearing/breeding housing according to claim 6, wherein the first longitudinal support flange and the second longitudinal flange leave a first gap at the first side of the housing and a second gap at the second side of the housing, for removal of the plurality of crates.

8. The insect rearing/breeding housing according to claim 7, wherein a cover plate is provided above and between the first gap and the second gap, which cover plate slopes downwards to at least one side along a moving direction of the plurality of crates.

9. The insect rearing/breeding housing according to claim 8, wherein a separating wall or plate extends from a top part of the cover plate to a top of the housing, separating the housing in two.

10. The insect rearing/breeding housing according to claim 8, wherein the cover plate is provided with a sealing for sealing towards an upper edge of the plurality of crates.

11. The insect rearing/breeding housing according to claim 1, wherein the housing is configured for supply of ventilating air through the housing bottom of the housing below the plurality of crates.

12. The insect rearing/breeding housing according to claim 1, wherein the housing comprises a fan or blower configured to suck ventilated air out at a top of the housing.

13. The insect rearing/breeding housing according to claim 1, wherein the housing comprises a top part with one or more openings, configured to supply ventilating air through the one or more openings.

14. The insect rearing/breeding housing according to claim 1, wherein the housing is configured for supply of ventilating air, alternating between being supplied through one or more openings at a top part of the housing and through the housing bottom from below the plurality of crates.

15. The insect rearing/breeding housing according to claim 3, wherein a first curtain is provided extending along a first side of the plurality of crates from the first longitudinal support flange and to at least an underside of the plurality of crates and a second curtain is provided extending along a second side of the plurality of crates from the second longitudinal support flange and to at least the underside of the plurality of crates.

16. The insect rearing/breeding housing according to claim 1, wherein the at least one ventilating opening is at a top of the housing is provided with a net or grate, preventing flies from entering the at least one ventilating opening.

17. The insect rearing/breeding housing according to claim 1, wherein an inspection window is provided at the end of the housing.

18. The insect rearing/breeding housing according to claim 1, wherein the crate insertion opening and/or the crate discharge opening is provided with a sealing for sealing towards an upper edge of the plurality of crates.

19. The insect rearing/breeding housing according to claim 1, wherein a conveyor is provided at the crate discharge opening for conveying discharged ones of the plurality of crates in a crosswise direction in relation to movement of the plurality of crates in the housing.

20. The insect rearing/breeding housing according to claim 19, wherein the conveyor is placed below the crate discharge opening at a middle portion of the housing at the housing bottom.
